(19) United States
(12) Patent Application Publication
(10) Pub. No.: US 2016/0195550 A1
(43) Pub. Date: Jul. 7, 2016

(54) METABOLISM OF SOD1 IN CSF

(71) Applicant: Washington University, St. Louis, MO (US)

(72) Inventors: Randall J. Bateman, St. Louis, MO (US); Timothy M. Miller, St. Louis, MO (US); Ling Munsell, St. Louis, MO (US)

(21) Appl. No.: 15/080,230

(22) Filed: Mar. 24, 2016

Related U.S. Application Data
(63) Continuation-in-part of application No. 14/352,560, filed on Apr. 17, 2014, now abandoned.
(60) Provisional application No. 61/547,890, filed on Oct. 17, 2011.

(51) Int. Cl. G01N 33/68

(52) U.S. Cl. CPC G01N 33/6896; G01N 2458/15; G01N 2333/90283; G01N 2800/28

(57) ABSTRACT

The invention relates to a method for measuring the metabolism of SOD1 in a subject in vivo.

### CROSS-REFERENCE TO RELATED APPLICATIONS

This application is a continuation-in-part of U.S. Ser. No. 14/352,560, filed Apr. 17, 2014, which claims the priority of PCT Application PCT/US2012/060597, filed Oct. 17, 2012, which claims the benefit of U.S. provisional application No. 61/547,890, filed Oct. 17, 2011, each of which is hereby incorporated by reference in its entirety.

### GOVERNMENTAL RIGHTS

This invention was made with government support under NS072584, NS074194 and AG030946 awarded by National Institutes of Health. The government has certain rights in this invention.

### FIELD OF THE INVENTION

The invention relates to a method for measuring the metabolism of SOD1 in a subject in vivo.

### REFERENCE TO SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821(f).

### BACKGROUND OF THE INVENTION

Amyotrophic lateral sclerosis (ALS) is a neurodegenerative disease marked by the progressive loss of motor neurons in the spinal cord and brain resulting in weakness, atrophy of skeletal muscles, loss of motor function, paralysis, and eventual death from respiratory failure 3-5 years post diagnosis. While 90% of ALS cases are sporadic, about 10% are dominantly-inherited. Of these familial cases, approximately 20% are due to dominant mutations in the enzyme Cu,Zn-superoxide dismutase 1 (SOD1), a homodimeric metalloenzyme that catalyzes the conversion of superoxide anion to hydrogen peroxide and molecular oxygen. Over 150 mutations have been characterized for this 153 amino acid protein that affect many aspects of its structure and function, such as catalytic activity, Cu and Zn binding sites, dimerization, intramolecular disulfide bond formation, and folding. As such, multiple hypotheses have been proposed to explain the mechanism behind mutant SOD1 toxicity, yet not one has been definitively proven. How this ubiquitously expressed protein imparts a selective toxicity to motor neurons of the spinal cord and primary motor cortex remains unknown.

Many neurodegenerative diseases are the result of the accumulation of mutant proteins. Although the expression of some of these proteins is largely limited to the CNS, others like SOD1 are ubiquitously expressed in all tissues of the body. Despite this universal expression, SOD1 mutations result in selective death of motor neurons. One attractive hypothesis is that the CNS handles misfolded, mutant proteins less effectively than other non-neuronal tissues. Indeed, global proteomics approaches using stable isotope labeling kinetics have shown that brain proteins have the lowest turnover rate, even if identical proteins or protein complexes are compared between tissues. These studies suggest that less efficient protein turnover in the CNS may set the stage for misfolded SOD1 accumulation that allows for pathology development. However, the comparison of turnovers rate of wild-type and mutant SOD1 between non-neuronal and neuronal tissues has never been studied and may yield valuable information regarding the tissue specificity of the disease.

A need exists, therefore, for a sensitive, accurate, and reproducible method for measuring the in vivo metabolism of biomolecules in the CNS. In particular, a method is needed for measuring the in vivo fractional synthesis rate and clearance rate of proteins associated with a neurodegenerative disease, e.g., the metabolism of SOD1 in ALS.

### SUMMARY OF THE INVENTION

One aspect of the invention provides methods for measuring the in vivo metabolism (e.g. the rate of synthesis, the rate of clearance) of SOD1.

Another aspect of the invention provides an in vitro method for measuring the in vivo metabolism of SOD1 in a subject who has received an amino acid labeled with a nonradioactive isotope (“a labeled amino acid”). The method comprises (a) detecting by mass spectrometry the amount of SOD1 labeled with a labeled amino acid (“labeled SOD1”) in a cerebral spinal fluid sample obtained from the subject, the cerebral spinal fluid sample comprising a labeled SOD1 fraction and a SOD1 fraction not labeled with the labeled amino acid (“unlabeled SOD1”); and (b) determining the ratio of labeled SOD1 to unlabeled SOD1, wherein the ratio of labeled SOD1 to unlabeled SOD1 is directly proportional to the metabolism of SOD1 in the subject. The sample may have been obtained either (i) between 20 and 35 days after the subject received the amino acid labeled with a nonradioactive isotope, or (ii) once a week for 2, 3, 4, or 5 weeks after the subject received the amino acid labeled with a nonradioactive isotope. Alternatively, the sample may have been obtained (i) between about 4 and about 40 days after the subject received the labeled amino acid labeled, (ii) between about 4 and about 30 days after the subject received the labeled amino acid labeled, or (iii) between about 4 and about 20 days after the subject received the labeled amino acid labeled. In addition, the sample may have been obtained (i) between about 20 and about 50 days after the subject received the labeled amino acid labeled, (ii) between about 20 and about 40 days after the subject received the labeled amino acid labeled, (iii) between about 20 and about 30 days after the subject received the labeled amino acid labeled, or (iv) (iii) between about 30 and about 40 days after the subject received the labeled amino acid labeled.

An additional aspect of the invention encompasses kits for measuring the in vivo metabolism of SOD1 in a subject.

### BRIEF DESCRIPTION OF THE FIGURES

The application file contains at least one photograph executed in color. Copies of this patent application publication with color photographs will be provided by the Office upon request and payment of the necessary fee.

FIG. 1A-C depicts images and graphs showing successful labeling of SOD1 WT (SOD1^{WT}) in cortex from transgenic rats. (A) Western blot showing immunoprecipitation with anti-SOD1 antibody from SOD1 WT rat cortex. (B) Formic acid elution and trypsin digestion of SOD1 from rat cortex. (C) Time-dependent incorporation of ¹³C₆-leucine in the SOD1 tryptic fragment TLVVHEK in brain and liver. FSR=fractional synthesis rate; T_{1/2}=SOD1 WT half life.

FIG. 2 depicts a graph showing tissue-specific differences in SOD1 G39A (SOD1^{G39A}) turnover. FCR=fractional clearance rate; T_{1/2}=SOD1 G39A half life; SC=spinal cord.

FIG. 3 depicts a graph showing mass spectrometry data of SOD1 from ¹³C₆-leucine labeled human CSF. Graphed on the Y-axis is the Area; graphed on the X-axis is time (hours). The graph shows that labeled human CSF samples showed a continued increase in labeled to unlabeled SOD1 ratio even at the latest time points.

FIG. 4 depicts a graph showing mass spectrometry data plotting H:L ratio along the Y-axis and time (hours) along the X-axis. Samples are human CSF samples and the squares represent the trypsin peptide fragment TLVVHEK_C13N14 (SEQ ID NO:1).

FIG. 5 depicts a graph showing a calibration curve of TLVVHEK_C13N14 (SEQ ID NO:1). The percent labeled versus the predicted value is shown with a linear regression line. Note the good linear fit, in addition to the low deviation.

FIG. 6 depicts a graph showing mass spectrometry data plotting H:L ratio along the Y-axis and time (hours) along the X-axis. Samples are human CSF samples. Red squares represent the trypsin peptide fragment HVGDLGNVTADK_C13N14 (SEQ ID NO:2) and blue squares represent TLVVHEK_C13N14 (SEQ ID NO:1).

FIG. 7 depicts a graph showing mass spectrometry data plotting H:L ratio along the Y-axis and time (hours) along the X-axis. Samples are human CSF samples and the squares represent the trypsin peptide fragment HVGDLGNVTADK_C13N14 (SEQ ID NO:2).

FIG. 8 depicts a graph showing a calibration curve of HVGDLGNVTADK_C13N14 (SEQ ID NO:2). The percent labeled versus the predicted value is shown with a linear regression line. Note the good linear fit, in addition to the low deviation.

FIG. 9 depicts a graph showing mass spectrometry data plotting Area ratio along the Y-axis and time (hours) along the X-axis. Samples are human CSF samples. Red squares represent the trypsin peptide fragment HVGDLGNVTADK_C13N14 (SEQ ID NO:2) and blue squares represent TLVVHEK_C13N14 (SEQ ID NO:1). Note; the squares overlap completely.

FIG. 10 depicts a diagram of a kinetic model of SOD1 labeling in humans. Plasma leucine represents a central compartment where tracer freely exchanges with all other measured compartments and whole body protein or is irreversibly lost from the system. From this central compartment, forward arrows indicate the forward exchange of tracer into each tissue compartment. The reverse arrows indicate tracer return and represent the FTR, expressed as pools per day, for each compartment. Dotted lines represent irreversible loss of tracer from the system. The diagram depicted is of a compartmental model accounting for plasma free ¹³C₆-leucine, CSF total protein, and CSF SOD1 over the full time course for human participants receiving a 10-day course of 13C6-leucine followed by a normal diet.

FIG. 11A-E depicts labeled SOD1 kinetics in healthy human subjects. (A) Schematic of the oral labeling paradigm. Participants were placed on a prepackaged low-leucine diet and administered ¹³C₆-leucine for 10 days, after which they resumed a normal diet. CSF and plasma were collected at the indicated time points, total protein was isolated from CSF and plasma and SOD1 was isolated from CSF, and isolated total protein and SOD1 were digested and analyzed by GC-MS and LC/tandem MS, respectively. (B-E) Data points with overlaid best-fit model curves (solid lines) of plasma-free ¹³C₆-leucine, CSF total protein, and CSF SOD1 for human (B) subject 1, (C) subject 2, (D) subject 3, and (E) subject 4. The legend for (B-D) is found in (E).

### DETAILED DESCRIPTION OF THE PREFERRED EMBODIMENTS

The present invention relates to determining the synthesis and clearance rates of SOD1. It also provides a method to assess whether a treatment is affecting the production or clearance rate of SOD1 in the CNS relevant to neurological and neurodegenerative diseases. The usefulness of this invention will be evident to those of skill in the art in that one may determine if a treatment alters the synthesis or clearance rate of SOD1. In particular, a significant advantage of the methods disclosed herein is the ability to independently measure both wildtype and mutant SOD1 species in the same biological sample, and even the same mass spectrometry sample, as single amino acid changes result in detectable changes in the m/z ratios of predicted peptides.

### I. Methods for Monitoring the In Vivo Metabolism of Neurally Derived Biomolecules

The current invention provides methods for measuring the in vivo metabolism of SOD1, including both wildtype SOD1 and mutant SOD1 species. By using this method, one skilled in the art may be able to study possible changes in the metabolism (synthesis and clearance) of SOD1 in a particular disease state. In addition, the invention permits the measurement of the pharmacodynamic effects of disease-modifying therapeutics in a subject.

In particular, this invention provides a method to label SOD1 as it is synthesized in the central nervous system in vivo; to collect a biological sample containing labeled and unlabeled SOD1; and a means to measure the labeling of SOD1 over time. These measurements may be used to calculate metabolic parameters, such as the synthesis and clearance rates within the CNS, as well as others.

### (a) Degenerative Diseases

Mutations in the gene for superoxide dismutase 1 (SOD1) account for 20% of dominantly inherited amyotrophic lateral sclerosis (ALS) cases. These genetic mutations ultimately produce SOD1 proteins with toxic properties. Currently, no treatment is available for familial ALS, but it is thought that inhibiting SOD1 production may limit the production of toxic SOD1 proteins and disease progression. Some treatments focus on lowering SOD1 protein levels in the brain and spinal cord. Decreased SOD1 in brain and spinal cord is reflected in decreased SOD1 in the cerebral spinal fluid (CSF). Thus, CSF SOD1 may be used as a biomarker for the treatment's effectiveness in reducing SOD1 synthesis.

Those of skill in the art will appreciate that, while ALS is the exemplary disease associated with SOD1, the invention is not limited to measuring SOD1 metabolism in subjects with ALS. It is envisioned that the method of the invention may be used to measure SOD1 metabolism in subjects with other neurological and neurodegenerative diseases, disorders, or processes related to SOD1 metabolism. It is also envisioned that the method of the invention may be used in healthy subjects.

It is envisioned that the in vivo metabolism of SOD1 will be measured in a human subject, and in certain embodiments, in a human subject with risk of developing ALS or in a human subject that has been diagnosed with ALS. Alternatively, the in vivo metabolism of SOD1 may be measured in other mammalian subjects. In one embodiment, the subject is a companion animal such as a dog or cat. In another embodiment, the subject is a livestock animal such as a cow, pig, horse, sheep or goat. In yet another alternative embodiment, the subject is a zoo animal. In another embodiment, the subject is a research animal such as a non-human primate or a rodent.

### (b) Labeled Moiety

Several different moieties may be used to label SOD1. Generally speaking, the two types of labeling moieties typically utilized in the method of the invention are radioactive isotopes and non-radioactive (stable) isotopes. In a preferred embodiment, non-radioactive isotopes may be used and measured by mass spectrometry. Preferred stable isotopes include deuterium ²H, ¹³C, ¹⁵N, ^{17 or 18}O, ^{33, 34, or 36}S, but it is recognized that a number of other stable isotope that change the mass of an atom by more or less neutrons than is seen in the prevalent native form would also be effective. A suitable label generally will change the mass of SOD1 under study such that it can be detected in a mass spectrometer. In one embodiment, the labeled moiety is an amino acid comprising a non-radioactive isotope (e.g., ¹³C). In another embodiment, the biomolecule to be measured is a nucleic acid, and the labeled moiety is a nucleoside triphosphate comprising a non-radioactive isotope (e.g., ¹⁵N). Alternatively, a radioactive isotope may be used, and the labeled biomolecules may be measured with a scintillation counter rather than a mass spectrometer. One or more labeled moieties may be used simultaneously or in sequence.

In a preferred embodiment, when the method is employed to measure the metabolism of a protein, the labeled moiety typically will be an amino acid. Those of skill in the art will appreciate that several amino acids may be used to provide the label of SOD1. Generally, the choice of amino acid is based on a variety of factors such as: (1) The amino acid generally is present in at least one residue of the protein or peptide of interest. (2) The amino acid is generally able to quickly reach the site of protein synthesis and rapidly equilibrate across the blood-brain barrier. Leucine is a preferred amino acid to label proteins that are synthesized in the CNS, as demonstrated in the Examples. (3) The amino acid ideally may be an essential amino acid (not produced by the body), so that a higher percent of labeling may be achieved. Non-essential amino acids may also be used; however, measurements will likely be less accurate. (4) The amino acid label generally does not influence the metabolism of the protein of interest (e.g., very large doses of leucine may affect muscle metabolism). And (5) availability of the desired amino acid (i.e., some amino acids are much more expensive or harder to manufacture than others). In one embodiment, ¹³C₆-phenylalanine, which contains six ¹³C atoms, is used to label SOD1. In a preferred embodiment, ¹³C₆-leucine is used to label SOD1.

There are numerous commercial sources of labeled amino acids, both non-radioactive isotopes and radioactive isotopes. Generally, the labeled amino acids may be produced either biologically or synthetically. Biologically produced amino acids may be obtained from an organism (e.g., kelp/seaweed) grown in an enriched mixture of ¹³C, ¹⁵N, or another isotope that is incorporated into amino acids as the organism produces proteins. The amino acids are then separated and purified. Alternatively, amino acids may be made with known synthetic chemical processes.

### (c) Administration of the Labeled Moiety

The labeled moiety may be administered to a subject by several methods. Suitable methods of administration include intravenously, intra-arterially, subcutaneously, intraperitoneally, intramuscularly, or orally. In some embodiments, the labeled moiety is a labeled amino acid, and the labeled amino acid is administered by intravenous infusion. In other embodiments, the labeled moiety is a labeled amino acid, and the labeled amino acid is administered as an intravenous bolus. In a preferred embodiment, the labeled moiety is a labeled amino acid and the labeled amino acid is orally ingested by the subject.

The amount (or dose) of labeled moiety can and will vary, as can the duration and frequency of administration. A labeled moiety may be administered to a subject one or more times a day (e.g. 1, 2, 3, 4, 5 or more times a day) on one or more days (e.g. 1, 2, 3, 4, 5, 6, 7, 8, 9, 10 or more days). The labeled moiety may be administered slowly over a period of time (e.g. IV infusion) or as a large single dose (i.e. IV bolus or orally) depending upon the type of analysis chosen (e.g., steady state or bolus/chase). A labeled moiety should be administered in a sufficient amount and for a sufficient duration so that labeled SOD1 is present in the biological sample in an amount that may be reliably quantified.

The amount of labeled SOD1 needed for reliable quantification is a function of the sensitivity of the quantitation method. Current mass spectrometry methods can measure as low as approximately 0.01-0.2% labeled SOD1, though about 1% to about 2% labeled SOD1 is preferred. However, these measurements are likely to improve (i.e. lower levels of labeled SOD1 may be measured) with advances in technology. One skilled in the art will appreciate that the percent labeled SOD1 needed for reliable quantification via other detection methods can readily be determined by routine experimentation, and labeling protocols can be modified based on the teachings herein.

In some embodiments, labeled amino acid may be intravenously or orally administered to a subject on one or more days. For example, labeled amino acid may be administered on 1, 2, 3, 4, 5, 6, 7, 8, 9, 10, or more days. The total daily dose of labeled amino acid may be divided into multiple smaller doses that are administered sequentially with little time elapsing between each dose, or the multiple doses may be administered at regular or irregular intervals throughout the day. The amount of time that elapses between each dose may be a few seconds, a few minutes, or a few hours. In preferred embodiments, a labeled amino acid is administered orally. In an exemplary embodiment, when a labeled amino acid is administered orally, it is provided to a subject as a drink. In an exemplary embodiment, labeled amino acid may be orally administered daily for 6, 7, 8, 9 or 10 days to achieve about 3% to about 4% labeled SOD1.

Those of skill in the art will appreciate that more than one label may be used in a single subject. This would allow multiple labeling of the same biomolecule and may provide information on the production or clearance of that biomolecule at different times. For example, a first label may be given to subject over an initial time period, followed by a pharmacologic agent (drug), and then a second label may be administered. In general, analysis of the samples obtained from this subject would provide a measurement of metabolism before and after drug administration, directly measuring the pharmacodynamic effect of the drug in the same subject.

Alternatively, multiple labels may be used at the same time to increase labeling of the biomolecule, as well as obtain labeling of a broader range of biomolecules.

### (d) Biological Sample

The method of the invention provides that a biological sample obtained from a subject be used to calculate the in vivo metabolism of the labeled SOD1. Suitable biological samples include, but are not limited to, bodily fluids or tissues in which SOD1 may be detected. For instance, in one embodiment, the bodily fluid is cerebral spinal fluid (CSF). In another embodiment, the biological sample is a tissue sample. For each type of biological sample, one of skill in the art should recognize that the half-life of SOD1 in the tissue should be determined in order to select sample collection times.

Cerebrospinal fluid may be obtained by lumbar puncture with or without an indwelling CSF catheter. Other types of samples may be collected by direct collection using standard good manufacturing practice (GMP) methods.

In general when the biomolecule under study is a protein, such as SOD1, the invention provides that a first biological sample may be taken from the subject prior to administration of the label to provide a baseline for the subject. Alternatively, when a first biological sample is not taken from the subject prior to administration of the label, an assumption may be made that the baseline sample has a normal isotopic distribution. After administration of the label, one or more samples may be taken from the subject. Biological samples may be taken over the course of more than two, three, four, five, six, seven, eight, nine or ten days. Alternatively, biological samples may be collected over the course of at least 2, 3, 4, 5, 6, 7, 8, 9, 10, 11, or 12 weeks. In general, biological samples obtained during the labeling phase may be used to determine rate of synthesis of SOD1, and biological samples taken during the clearance phase may be used to determine the clearance rate of SOD1. In addition, biological samples obtained at various times throughout the SOD1 labeling curve may be used to determine other aspects of SOD1 metabolism (e.g. labeled SOD1 peak time, labeled SOD1 peak amount, absolute quantitation, relative labeling, fractional turnover rate, etc.). As will be appreciated by those of skill in the art, the number of samples and when they may be taken generally will depend upon a number of factors such as: the type of analysis, type of administration, the protein of interest, the rate of metabolism, the type of detection, etc. Different tissues and different mutations in SOD1 may require different collection times, as discussed in further detail in the examples.

In one embodiment, the biomolecule is SOD1 and samples of CSF are taken over the course of several days. As the half-life of SOD1 in CSF is generally thought to be greater than 3 weeks, CSF samples may be taken over the course of more than three, four, five, six, seven, eight, nine, or ten days. In some embodiments, one or more samples may be collected once a week for at least 2, 3, 4, 5, 6, 7, 8, or 9 weeks. In other embodiments, samples may be collected on at least one time point prior to the half-life of SOD1 in CSF, and at least one time point greater than the half-life of SOD1. In a particular embodiment, at least one sample may be collected between about 20 days and about 35 days. In another embodiment, at least one sample may be collected between about 20 days and about 30 days. In yet another embodiment, at least one sample may be collected between about 25 days and about 35 days. In this context, ‘about’ means±1 day.

In another embodiment, a biological sample is a CSF sample and at least 1, 2, 3, 4, 5, 6, 7, 8, 9, or 10 samples are collected between day 0 and day 40, between day 1 and day 40, between day 2 and day 40, between day 3 and day 40, between day 4 and day 40, or between day 5 and day 40. For example, at least 1, 2, 3, 4, 5, 6, 7, 8, 9, or 10 CSF samples may be collected on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or day 40, or any combination thereof. In other embodiments, a biological sample is a CSF sample and at least 1, 2, 3, 4, 5, 6, 7, 8, 9, or 10 samples are collected between day 0 and day 35, between day 1 and day 35, between day 2 and day 35, between day 3 and day 35, between day 4 and day 35, or between day 5 and day 35. In other embodiments, a biological sample is a CSF sample and at least 1, 2, 3, 4, 5, 6, 7, 8, 9, or 10 samples are collected between day 0 and day 30, between day 1 and day 30, between day 2 and day 30, between day 3 and day 30, between day 4 and day 30, or between day 5 and day 30. In other embodiments, a biological sample is a CSF sample and at least 1, 2, 3, 4, 5, 6, 7, 8, 9, or 10 samples are collected between day 0 and day 25, between day 1 and day 25, between day 2 and day 25, between day 3 and day 25, between day 4 and day 25, or between day 5 and day 25. In other embodiments, a biological sample is a CSF sample and at least 1, 2, 3, 4, 5, 6, 7, 8, 9, or 10 samples are collected between day 5 and 40, between day 5 and 35, between day 5 and day 30, between day 5 and 25. In other embodiments, a biological sample is a CSF sample and at least 1, 2, 3, 4, 5, 6, 7, 8, 9, or 10 samples are collected between day 10 and 40, between day 10 and 35, between day 10 and day 30, between day 10 and 25. In embodiments where the biological sample is a CSF sample, sample collection between day 0 and day 40, preferably between day 5 and day 40 or between day 10 and day 40, may be used to determine the rate of labeled SOD1 production or metabolic parameters associated with SOD1 production.

In some embodiments, a biological sample is a CSF sample and at least 1, 2, 3, 4, 5, 6, 7, 8, 9, or 10 samples are collected between day 25 and day 45. For example, at least 1, 2, 3, 4, 5, 6, 7, 8, 9, or 10 CSF samples may be collected on day 25, 26, 27, 28, 29, 30, 31, 32, 33, 34, 35, 36, 37, 38, 39, 40, 41, 42, 43, 44, or day 45, or any combination thereof. In other embodiments, a biological sample is a CSF sample and at least 1, 2, 3, 4, 5, 6, 7, 8, 9, or 10 samples are collected between day 25 and day 40, day 25 and day 35, or between day 25 and day 30. In other embodiments, a biological sample is a CSF sample and at least 1, 2, 3, 4, 5, 6, 7, 8, 9, or 10 samples are collected between day 30 and day 45, between day 30 and day 40, or between day 30 and day 35. In other embodiments, a biological sample is a CSF sample and at least 1, 2, 3, 4, 5, 6, 7, 8, 9, or 10 samples are collected between day 35 and day 45, or between day 35 and day 40. In embodiments where the biological sample is a CSF sample, sample collection between day 25 and day 45 may be used to determine the peak of labeled SOD1 production or metabolic parameters associated labeled SOD1 peak production (e.g. time to peak, peak height, etc.). When the peak of labeled SOD1 production is reasonably known, sample collection between day 25 and day 45 may be also used to determine metabolic parameters associated with labeled SOD1 production and labeled SOD1 clearance (e.g. samples collected before the peak of labeled SOD1 production may be used to calculate metabolic parameters associated with labeled SOD1 production and samples collected after the peak of labeled SOD1 production may be used to calculate metabolic parameters associated with labeled SOD1 clearance.)

In some embodiments, a biological sample is a CSF sample or a blood sample and at least 1, 2, 3, 4, 5, 6, 7, 8, 9, or 10 samples are collected between day 40 and day 100. For example, at least 1, 2, 3, 4, 5, 6, 7, 8, 9, or 10 CSF samples may be collected on day 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or day 100. In some embodiments, a biological sample is a CSF sample and at least 1, 2, 3, 4, 5, 6, 7, 8, 9, or 10 samples are collected between day 40 and day 90, between day 40 and day 80, between day 40 and day 70, or between day 40 and day 60. In other embodiments, a biological sample is a CSF sample and at least 1, 2, 3, 4, 5, 6, 7, 8, 9, or 10 samples are collected between day 50 and day 100, between day 50 and day 90, between day 50 and day 80, between day 50 and day 70, or between day 50 and day 60. In yet other embodiments, a biological sample is a CSF sample and at least 1, 2, 3, 4, 5, 6, 7, 8, 9, or 10 samples are collected between day 60 and day 100, between day 60 and day 90, between day 60 and day 85, between day 60 and day 80, or between day 60 and day 70. In still other embodiments, a biological sample is a CSF sample and at least 1, 2, 3, 4, 5, 6, 7, 8, 9, or 10 samples are collected between day 70 and day 100, between day 70 and day 95, between day 70 and day 90, between day 70 and day 85, or between day 70 and day 80. In embodiments where the biological sample is a CSF sample, sample collection between day 40 and day 100 may be used to determine the rate of labeled SOD1 production or metabolic parameters associated with SOD1 production.

In other embodiments, the biomolecule is SOD1 and samples of liver tissue are taken over the course of several days. For instance, one or more samples may be taken at about 14, 15, 16, 17, 18, 19, or 20 days. In another embodiment, the biomolecule is SOD1 and samples of brain tissue are taken over the course of several days. For instance, one or more samples may be taken at about 25, 26, 27, 28, 29, 30, 31 or 32 days.

Generally speaking, at least two samples should be collected. In some embodiments, two, three, or four samples may be collected. In certain embodiments, more than four samples may be collected.

### (e) Detection

The present invention provides that detection of the amount of labeled SOD1 and the amount of unlabeled SOD1 in the biological samples may be used to determine the ratio of labeled SOD1 to unlabeled SOD1. Generally, the ratio of labeled to unlabeled SOD1 is directly proportional to the metabolism of SOD1. Suitable methods for the detection of labeled and unlabeled SOD1 can and will vary according to the form of SOD1 under study and the type of labeled moiety used to label it. If the biomolecule of interest is a protein and the labeled moiety is a non-radioactively labeled amino acid, then the method of detection typically should be sensitive enough to detect changes in mass of the labeled protein with respect to the unlabeled protein. In a preferred embodiment, mass spectrometry is used to detect differences in mass between the labeled and unlabeled SOD1. In one embodiment, gas chromatography mass spectrometry is used. In an alternate embodiment, MALDI-TOF mass spectrometry is used. In a preferred embodiment, high-resolution tandem mass spectrometry is used.

Additional techniques may be utilized, alone or in combination, to separate the protein of interest from other proteins and biomolecules in the biological sample. As an example, detergent soluble SOD1 may be separated from total SOD1 by extracting the biological sample with an extraction buffer that contains detergent. As another example, immunoprecipitation may be used to isolate and partially, or completely, purify SOD1 before it is analyzed by mass spectrometry. A skilled artisan will appreciate that the choice of antibody will greatly influence the form(s) of SOD1 isolated from the biological tissue. As a non-limiting example, anti-SOD1 antibodies may be used to immunoprecipitate misfolded SOD1, properly folded SOD1, or total SOD1.

Other methods of separating or concentrating SOD1 may be used alone or in combination with immunoprecipitation and/or detergent solubilization. For example, chromatography techniques may be used to separate SOD1 (or fragments thereof) by size, hydrophobicity or affinity. In particular, techniques linking a chromatographic step with mass spectrometry may be use. In an exemplary embodiment, SOD1 is immunoprecipitated and then analyzed by a liquid chromatography system interfaced with a tandem MS unit equipped with an electrospray ionization source (LC-ESI-tandem MS).

Labeled and unlabeled SOD1 may also be cleaved into smaller peptides prior to detection. For instance, SOD1 may be enzymatically cleaved with a protease to create several small peptides. Suitable proteases include, but are not limited to, trypsin and Gluc. In an exemplary embodiment, labeled and unlabeled tau is completely or partially purified from a biological sample, enzymatically cleaved with a protease, and then analyzed by a liquid chromatography system interfaced with a high-resolution tandem MS unit. In another exemplary embodiment, labeled and unlabeled tau is enzymatically cleaved with a protease in a composition comprising a biological sample, the SOD1 fragments are completely or partially purified from the biological sample, and then analyzed by a liquid chromatography system interfaced with a high-resolution tandem MS unit.

The invention also provides that multiple proteins or peptides in the same biological sample may be measured simultaneously. That is, both the amount of unlabeled and labeled protein (and/or peptide) may be detected and measured separately or at the same time for multiple proteins. As such, the invention provides a useful method for screening changes in synthesis and clearance of proteins on a large scale (i.e. proteomics/metabolomics) and provides a sensitive means to detect and measure proteins involved in the underlying pathophysiology.

### (f) Metabolism Analysis

Once the amount of labeled and unlabeled SOD1 has been detected in a biological sample, the relative labeling of SOD1 may be calculated. As used herein, “relative labeling” may refer to a ratio of labeled to unlabeled SOD1 or the percent of labeled SOD1. The amount of labeled SOD1, unlabeled SOD1, or the relative labeling of SOD1 may be used to calculate one or more additional parameter of SOD1 metabolism. Non-limiting examples of suitable metabolic parameters include the fractional synthesis rate, the fractional clearance rate, absolute synthesis rate, absolute clearance rate, fractional turnover rate, half-life, time to peak height, peak height, etc. Methods for calculating these parameters are known in the art, and those of skill in the art will be familiar with the first order kinetic models of labeling that may be used with the method of the invention. In addition, other parameters, such as lag time and isotopic tracer steady state, may be determined and used as measurements of SOD1's metabolism and physiology. Also, modeling may be performed on the data to fit multiple compartment models to estimate transfer between compartments. Of course, the type of mathematical modeling chosen will depend on the individual protein synthetic and clearance parameters (e.g., one-pool, multiple pools, steady state, non-steady-state, compartmental modeling, etc.). Further details regarding a suitable model are described in the examples.

The amount of labeled SOD1 in a biological sample at a given time reflects the metabolism of SOD1, including the synthesis rate (i.e. production) or the clearance rate (i.e. removal or destruction). The invention provides that the synthesis of SOD1 is typically based upon the rate of increase of the labeled/unlabeled protein ratio over time (i.e., the slope, the exponential fit curve, or a compartmental model fit defines the rate of SOD1 synthesis). For these calculations, a minimum of one sample is typically required (one could estimate the baseline label), two are preferred, and multiple samples are more preferred to calculate an accurate curve of the uptake of the label into the protein (i.e., the synthesis rate). Conversely, after the administration of labeled amino acid is terminated, the rate of decrease of the ratio of labeled to unlabeled protein typically reflects the clearance rate of that protein. For these calculations, a minimum of one sample is typically required (one could estimate the baseline label), two are preferred, and multiple samples are more preferred to calculate an accurate curve of the decrease of the label from the protein over time (i.e., the clearance rate). The amount of labeled protein in a biological sample at a given time may be expressed as percent per hour or the mass/time (e.g., mg/hr) of the protein in the subject, or other suitable unit.

In an exemplary embodiment, as illustrated in the examples, the in vivo metabolism of SOD1 is measured by administering labeled leucine to a subject over 9 hours and collecting at least one biological samples at a time point greater than 4 days after administration of the label. The biological sample may be collected from CSF. The amount of labeled and unlabeled SOD1 in the biological samples is typically determined by immunopreciptitation followed by LC-ESI-tandem MS. From these measurements, the ratio of labeled to unlabeled SOD1 may be determined, and this ratio permits the determination of metabolism parameters, such as rate of synthesis and rate of clearance of SOD1.

In another exemplary embodiment, as illustrated in the examples, the in vivo metabolism of SOD1 can be measured by orally administering non-radioactively labeled leucine (e.g. ¹³C₆-leucine) to a subject for ten days and collecting at least one biological samples at a time point greater than 4 days after administration of the label. The biological sample may be collected from CSF. The amount of labeled and unlabeled SOD1 in the biological samples is typically determined by immunopreciptitation followed by LC-ESI-tandem MS. From these measurements, the ratio of labeled to unlabeled SOD1 may be determined, and this ratio permits the determination of metabolism parameters, such as rate of synthesis and rate of clearance of SOD1.

### II. Kits for Monitoring the Progression or Treatment of Neurological and Neurodegenerative Diseases

The current invention provides kits for measuring SOD1 or monitoring the progression or treatment of a neurological or neurodegenerative disease associated with SOD1 by measuring the in vivo metabolism of a central nervous system-derived protein in a subject. Generally, a kit comprises a labeled amino acid, means for administering the labeled amino acid, means for collecting biological samples over time, and instructions for detecting and determining the ratio of labeled to unlabeled SOD1 so that a metabolic index may be calculated. The metabolic index then may be compared to a metabolic index of a normal, healthy individual or compared to a metabolic index from the same subject generated at an earlier time. In a preferred embodiment, the kit comprises ¹³C₆-leucine or ¹³C₆-phenylalanine, the protein to be labeled is SOD1, and the disease to be assessed is ALS.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Clearance rate” refers to the rate at which the biomolecule of interest, such as SOD1, is removed.

“Fractional clearance rate” or FCR is calculated as the natural log of the ratio of labeled biomolecule, such as labeled SOD1, over a specified period of time.

“Fractional synthesis rate” or FSR is calculated as the slope of the increasing ratio of labeled biomolecule, such as labeled SOD1, over a specified period of time divided by the predicted steady state value of the labeled precursor.

“Fractional turnover rate” or FTR is the rate of irreversible loss of a biomolecule, such as SOD1, from the CNS, and is the sum of losses to CSF and other loss pathways (e.g. local tissue uptake, proteolysis, deposition).

“Isotope” refers to all forms of a given element whose nuclei have the same atomic number but have different mass numbers because they contain different numbers of neutrons. By way of a non-limiting example, ¹²C and ¹³C are both stable isotopes of carbon.

“Lag time” generally refers to the delay of time from when a biomolecule, such as SOD1, is first labeled until the labeled biomolecule is detected.

“Metabolism” refers to any combination of the synthesis, transport, breakdown, modification, or clearance rate of a biomolecule, such as SOD1.

“Metabolic index” refers to a measurement comprising the fractional synthesis rate (FSR) and the fractional clearance rate (FCR) of the biomolecule of interest.

“Neurally derived cells” includes all cells within the blood-brain-barrier including neurons, astrocytes, microglia, choroid plexus cells, ependymal cells, other glial cells, etc.

“Start of labeling” refers to the time at which labeling begins, i.e. time=zero. For SOD1 labeling protocols that require administration of a label on multiple days, the “start of labeling” refers to the first time label is administered.

“Steady state” refers to a state during which there is insignificant change in the measured parameter over a specified period of time.

“Synthesis rate” refers to the rate at which a biomolecule, such as SOD1, is synthesized.

In metabolic tracer studies, a “stable isotope” is a nonradioactive isotope that is less abundant than the most abundant naturally occurring isotope.

“Subject” as used herein means a living organism having a central nervous system. In particular, the subject is a mammal. Suitable subjects include research animals, companion animals, farm animals, and zoo animals. The preferred subject is a human.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Stable Isotope Labeling Kinetics in a Rat Model of ALS

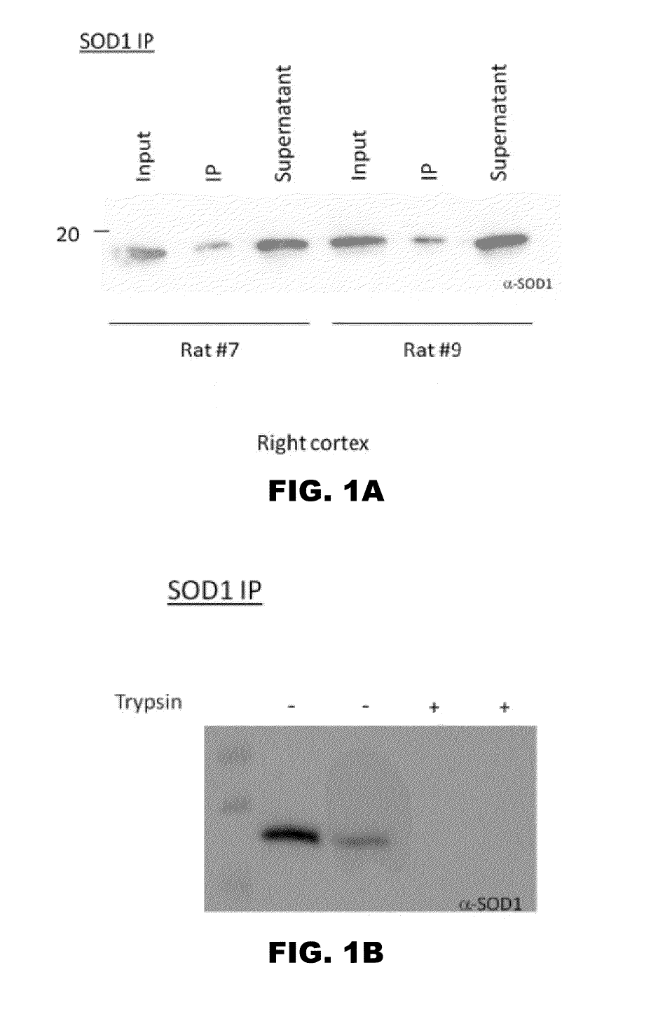

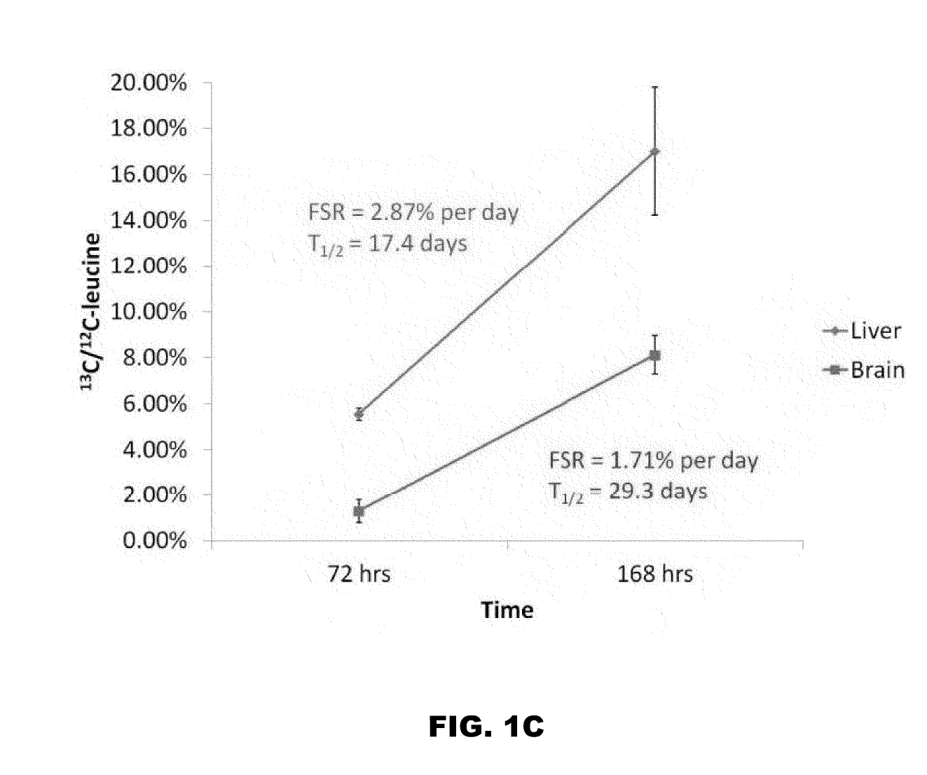

Transgenic rats overexpressing human SOD1 WT were fed a leucine-free chow for two weeks prior to labeling with ¹³C-leucine in order to acclimate the rats to the novel diet. During this acclimation period, normal ¹²C₆-leucine (100 mg/day) was provided in the drinking water, sweetened slightly with sucrose. After the acclimation period, the drinking water was replaced with ¹³C₆-leucine (100 mg/day) in order to label the animals. Data presented in FIGS. 1A and 1B show that a 7 day labeling period results in sufficient SOD1 label in brain and liver. After the 7 day labeling period, the animals were chased with normal ¹²C₆-leucine, after which animals were sacrificed at specific time points, perfused with PBS/heparin, and brain, spinal cord, liver, and kidney was harvested, flash frozen in liquid nitrogen, and stored at −80° C.

SOD1 was immunoprecipitated from tissue lysates using an anti-SOD1 mouse monoclonal antibody (Sigma) covalently linked to magnetic Dynabeads (Invitrogen). Briefly, tissues are thawed on ice and mechanically homogenized using a hand blender in NP-40 lysis buffer (1% NP-40, 150 mM Tris, protease inhibitors). SOD1 is immunoprecipitated from 500 μg of total protein using 50 μL of anti-SOD1 crosslinked beads overnight. The beads are washed three times in PBS and SOD1 eluted from the beads with 50 μL of formic acid. The formic acid eluent is lyophilized via vacuum and resuspended in 25 mM NaHCO₃ buffer. 400 ng of sequencing grade trypsin is added to the samples and digestion allowed to proceed at 37° C. for 18 hours. Samples are again lyophilized via vacuum and resuspended in 20 μL of 0.05% formic acid in preparation for the LC/MS run (Xevo). The ratio of labeled to unlabeled SOD1 is determined by comparing the area under the curve for the peptide TLVVHEK (SEQ ID NO:1) with or without ¹³C₆-leucine, respectively. From these labeling data, the fractional synthesis rate (FSR) and fractional clearance rate (FCR) can be calculated, depending on if the tissues were taken during the labeling or chase period, respectively.

### Example 2

### Tissue-Specific FSR for SOD1 WT Transgenic Rats

As can be seen in FIG. 1C, SOD1 from two different tissues (brain and liver) was immunoprecipitated, digested, and analyzed from transgenic rats overexpressing human SOD1 WT. It is clear from the data that a tissue-specific difference exists between the brain and liver. Specifically, brain synthesizes SOD1 at a rate that is 60% of liver. As FSR and FCR are intrinsically linked to maintain protein steady-state levels, the half-life of SOD1 WT in the brain and liver can be estimated to be 29.3 and 17.4 days, respectively. The long half-life for brain is in agreement with a prior study looking at SOD1-YFP half-life in the spinal cords of transgenic mice.

### Example 3

### Tissue-Specific FCR for SOD1 G93A Transgenic Rats

Transgenic rats overexpressing human SOD1 G93A were fed a leucine-free chow for two weeks prior to labeling with ¹³C₆-leucine in order to acclimate the rats to the novel diet. During this acclimation period, normal ¹²C₆-leucine (100 mg/day) was provided in the drinking water, sweetened slightly with sucrose. After the acclimation period, the drinking water was replaced with ¹³C₆-leucine (100 mg/day) in order to label the animals. After a 7 day labeling period, the animals were chased with normal ¹²C₆-leucine, after which animals were sacrificed at 3-days and 10-days post-label, perfused with PBS/heparin, and brain, spinal cord, liver, and kidney was harvested, flash frozen in liquid nitrogen, and stored at −80° C.

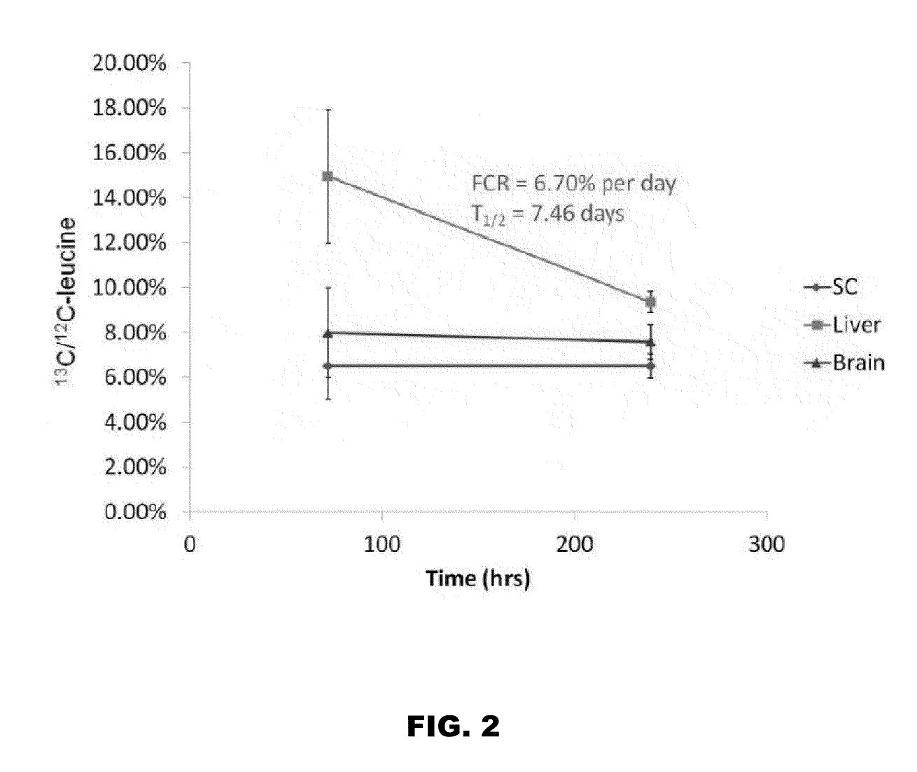

As shown in FIG. 2, the FCR of G93A in liver is determined to be 6.70% per day, which translates into a half-life of 7.46 days. Brain and spinal cord were not significantly different enough between time points to have confidence in calculating an FCR, but the data show that these tissues degrade SOD1 G93A much slower than in liver. This suggests that the spacing between collection points, for certain CSF embodiments, should be greater than 3 days.

### Example 4

### Stable Isotope Labeling of SOD1 in Humans

CSF samples were previously obtained from healthy volunteers after administration of a stable isotope-labeled amino acid (¹³C₆-leucine). Briefly, participants had 2 Ws and one lumbar catheter placed. In one IV, ¹³C₆-labeled leucine was infused for 9 or 12 hours. Each hour, plasma and CSF were obtained through the other IV and the lumbar catheter, respectively. Samples were taken at 1-hour time intervals for 36 hours. Further details can be found in Batemen et. al. Nat. Med. 2006, which is incorporated by reference herein.

Monoclonal anti-superoxide dismutase antibody was purified using a Protein A IgG Purification Kit (Pierce) and coupled to magnetic beads using a M-270 Epoxy Dynabeads Antibody Coupling Kit (Invitrogen). SOD1 was immunoprecipitated from CSF samples using the anti-SOD1 antibody coupled to the magnetic beads. The immunoprecipitated SOD1 samples were then digested with trypsin for 18 hours at 37° C. and run on LC/MS.

After trypsin digestion, two SOD1 peptides, TLVVHEK (SEQ ID NO:1) and HVGDLGNVTADK (SEQ ID NO:2), showed the highest intensity from mass spectrometry and were used for subsequent studies and analysis. Mass spectrometry of ¹³C₆-leucine labeled human CSF samples at time points, 0 hrs, 6 hrs, 12 hrs, 17 hrs, 18 hrs, 24 hrs, 30 hrs, and 36 hrs was performed.

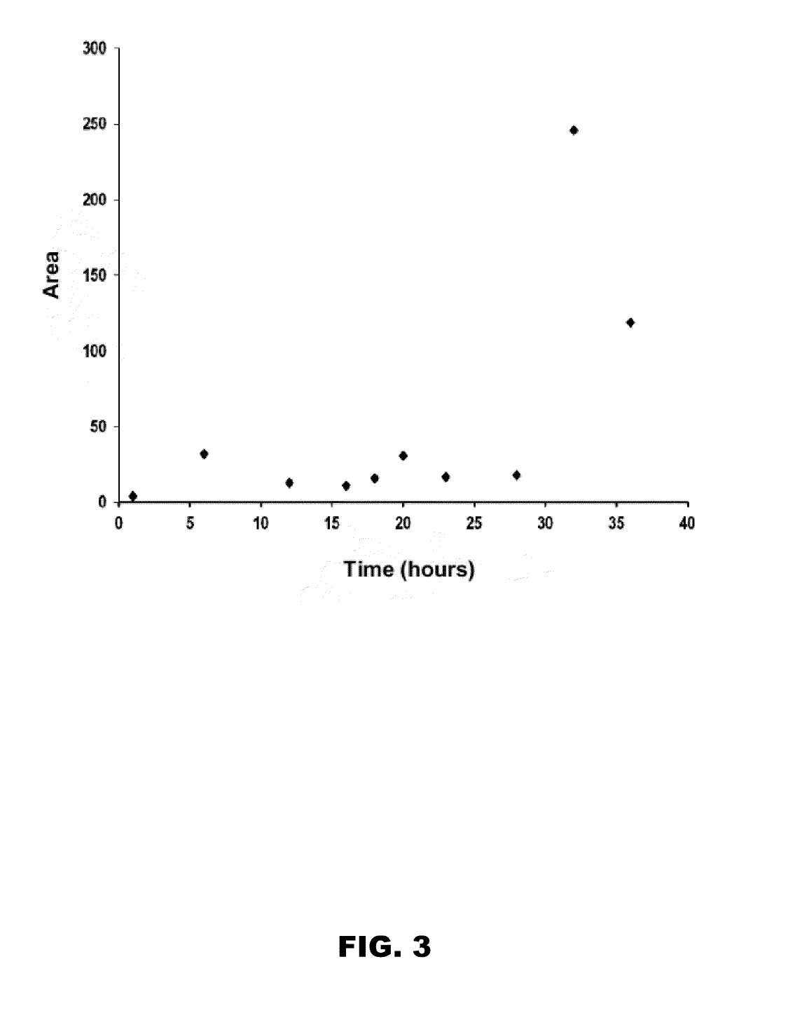

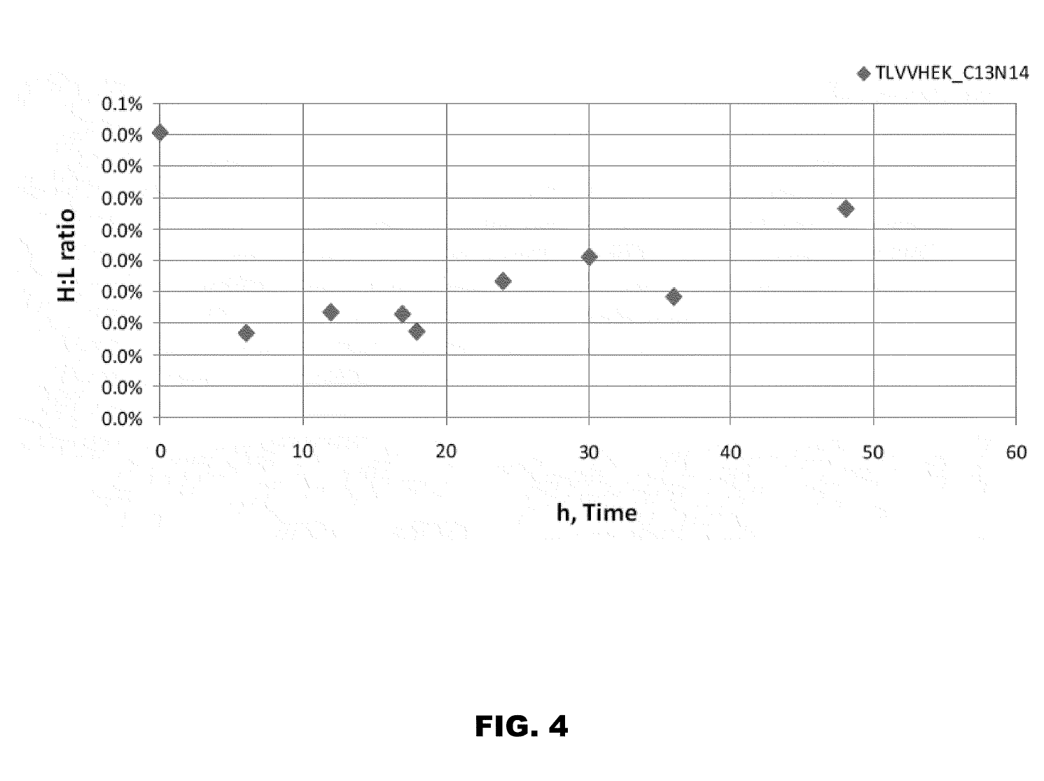

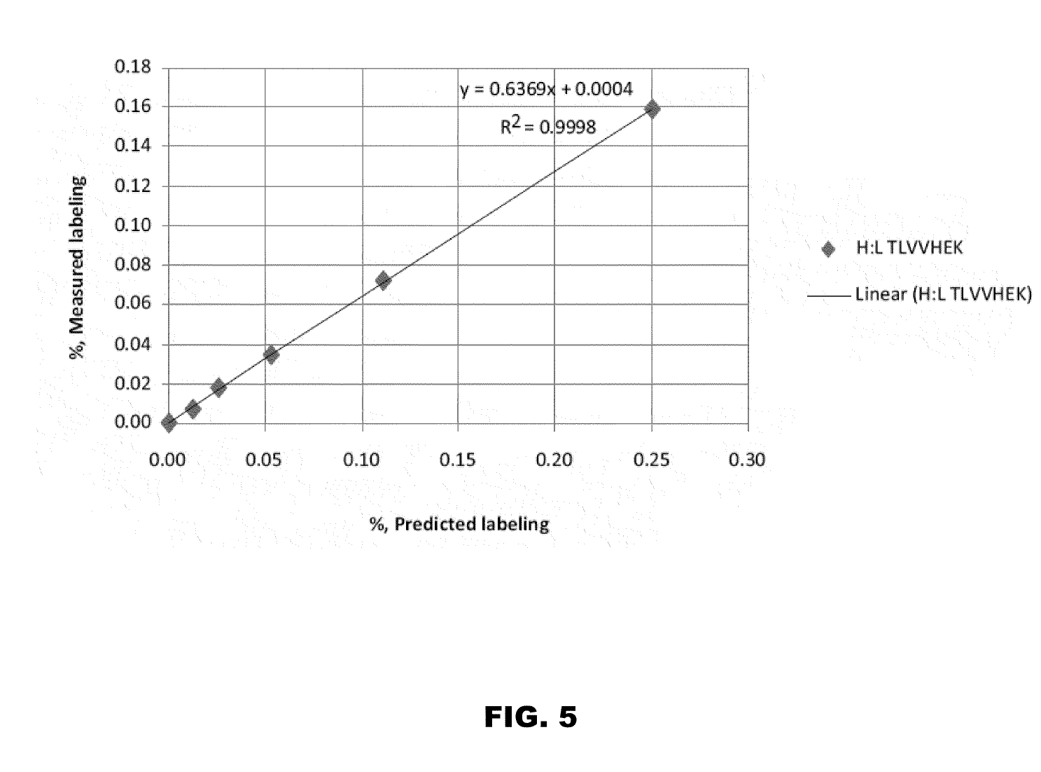

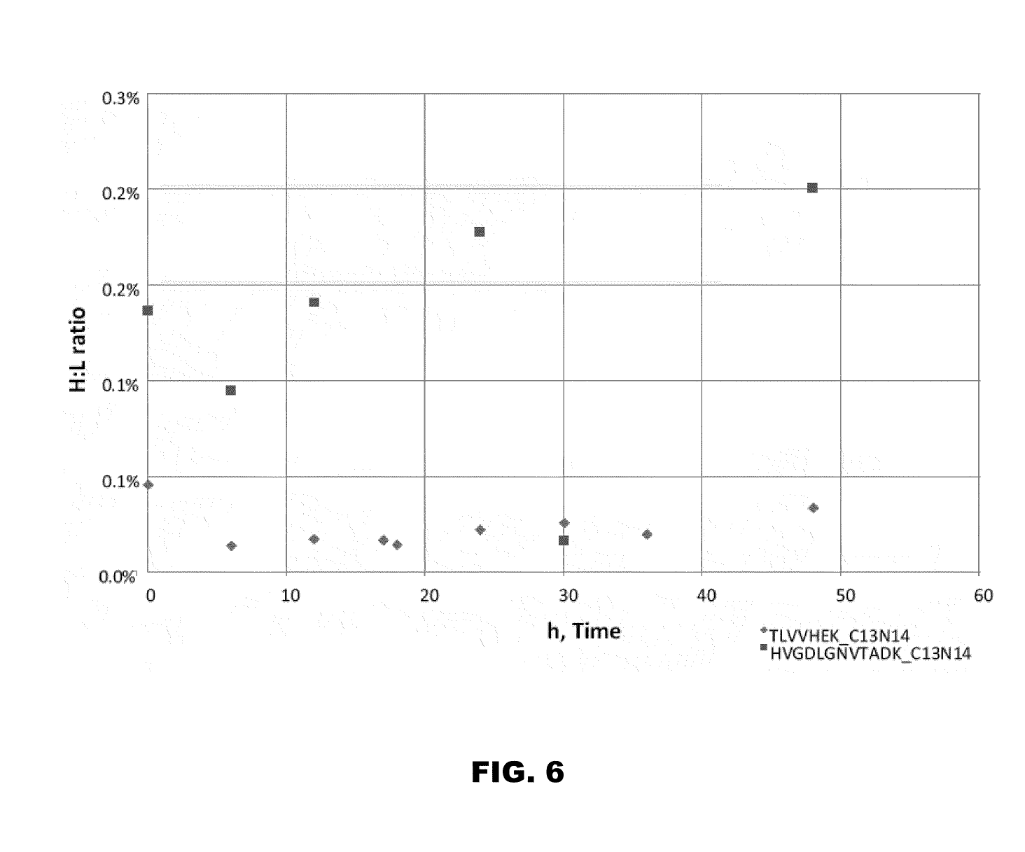

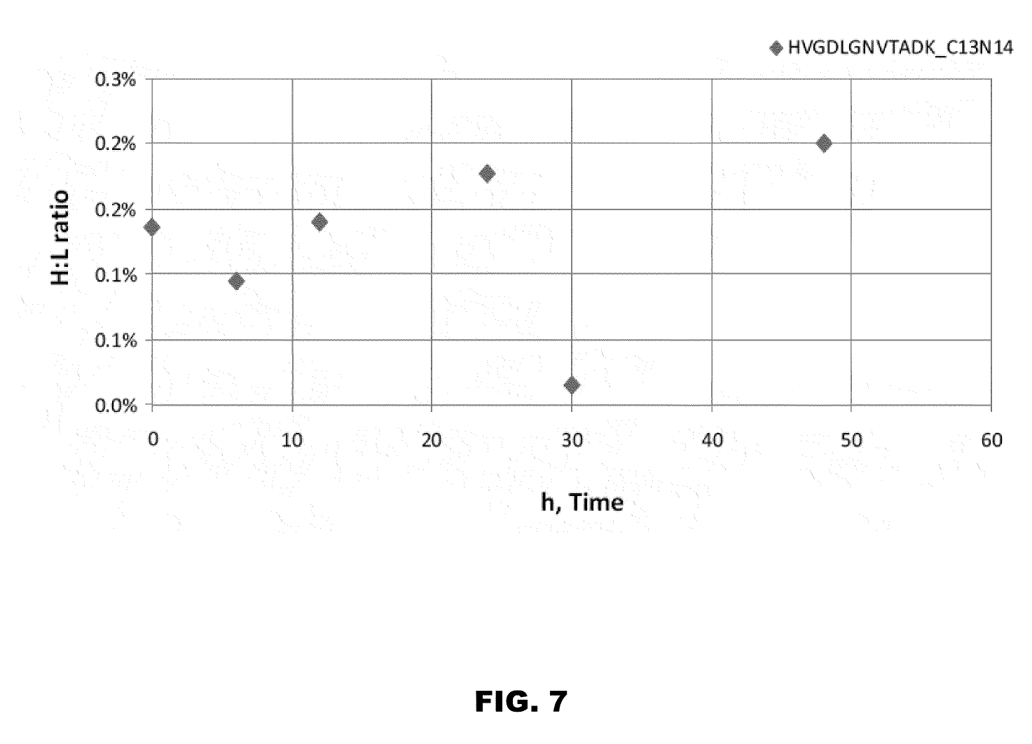

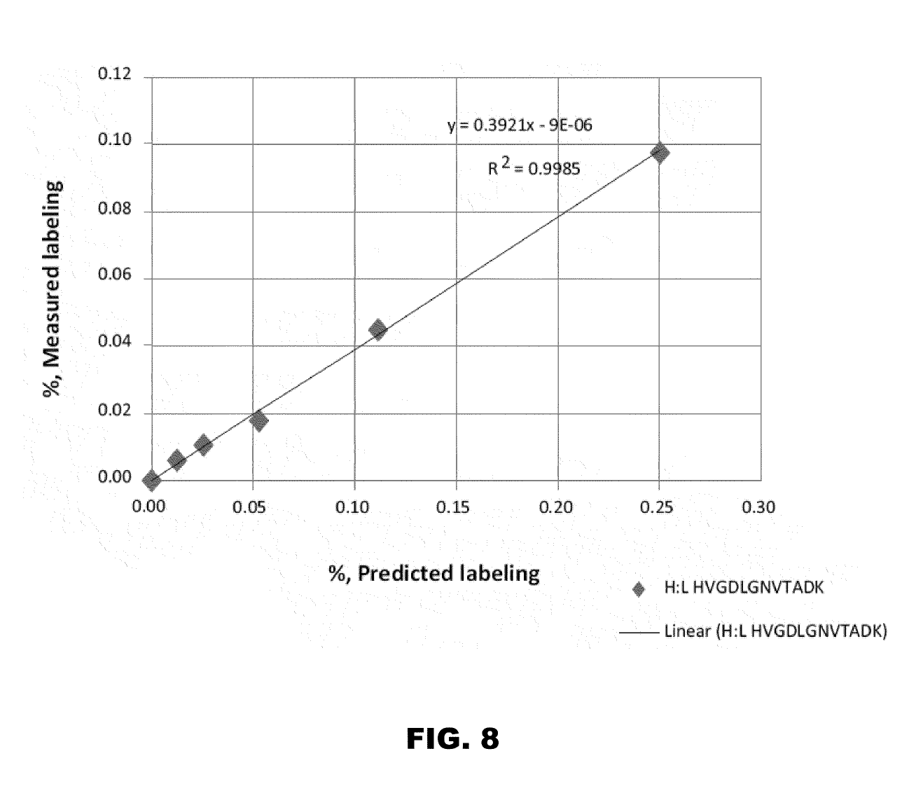

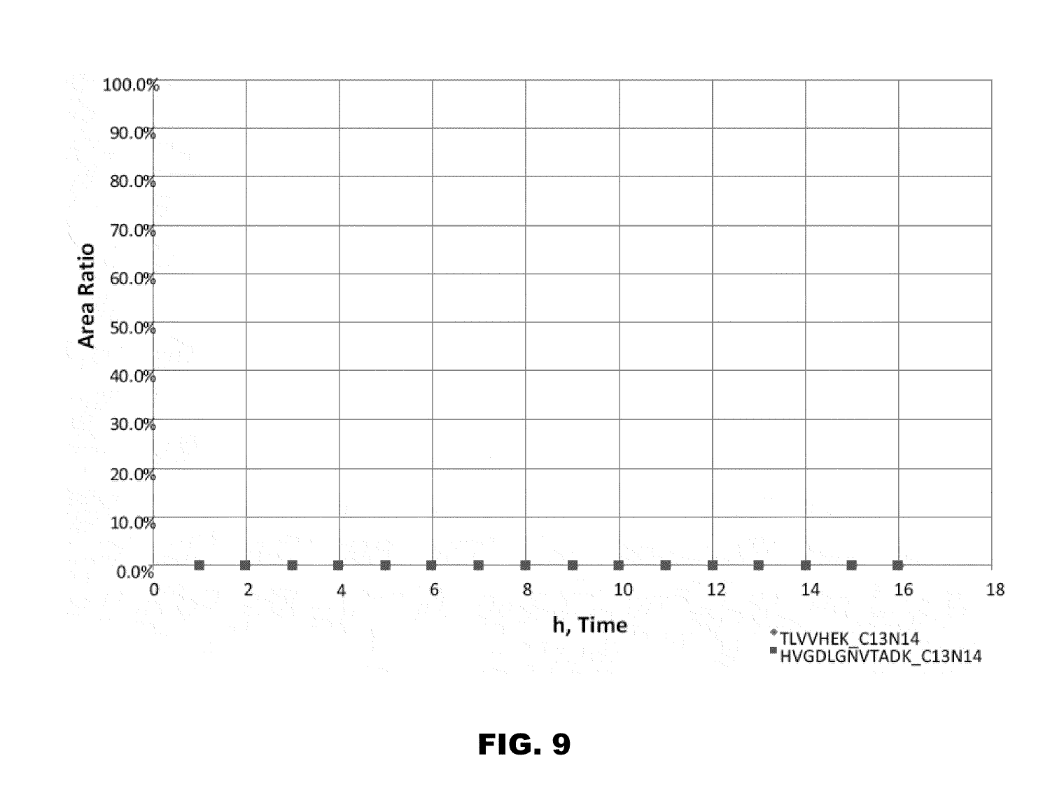

Previously, this stable isotype labeling method was performed on an extracellular protein, amyloid beta. Because SOD1 is an intracellular protein, it is likely that the rate of SOD1 excretion into the CSF is much slower than anticipated or that the half-life of SOD1 is very long (many days to weeks). As shown in FIG. 3, it was not possible to detect a peak in the strength of the mass spec signal for the peptides of interest within the 36 hour time point.

### Example 5

### Stable Isotope Labeling of SOD1 in Humans

In these human studies, human participants consumed a controlled leucine-containing diet supplemented with ¹³C₆-leucine powder for 10 days. The controlled leucine diet (approximately 2,000 mg leucine per day) was prepared by dieticians in the Washington University Research Kitchen, handed to the subjects, and consumed at home. Food intake was monitored by a self-reported food journal. Participants consumed ¹³C₆-leucine (Cambridge Isotope Laboratories CLM-2262) after dissolving 330 mg ¹³C₆-leucine and Kool-Aid-flavored powder in 120 ml tap water 3 times per day (total daily dose=990 mg). During the ¹³C₆-leucine-labeling period, overnight fasting blood was collected on days 1 and 10 of the research meal plan. On day 11, participants resumed consumption of their habitual diets. CSF (via lumbar puncture) and venous blood samples were collected approximately 14, 28, 42, and 67-84 days after ¹³C₆-leucine-labeling was initiated (actual time points differed slightly among subjects). Approximately 20 to 25 ml of CSF was drawn with each lumbar puncture.

Plasma and CSF samples were processed as described below. Blood was centrifuged at 1,800 g for 10 minutes, and the serum was aliquoted into low-binding 1.5-ml tubes (Ambion AM12450), frozen on dry ice, and stored at −80° C. CSF (20-25 ml) was centrifuged at 1,000 g for 10 minutes at 4° C., and 1 ml was aliquoted into several low-binding 1.5-ml tubes, frozen on dry ice, and stored at −80° C. Immunoprecipitation of SOD1 from 1 ml CSF was carried out by adding 50 μl anti-SOD1 cross-linked M-270 Dynabeads, protease inhibitors, and Tween (final concentration 0.1%) and rotating the tubes overnight at 4° C. SOD1 was eluted from the beads (99% 50 μl formic acid). The formic acid eluent was transferred to a new polypropylene tube and lyophilized via speed vacuum (Labconco CentriVap), resuspended with 100% methanol, lyophilized again via speed vacuum, and resuspended in 25 μl of 25 mM NaHCO₃ buffer (pH 8.8). 600 ng of sequencing-grade endoproteinase Glu-C(Roche) was added to each sample and digestion was allowed to proceed at 25° C. for 17 hours. Samples were again lyophilized via vacuum and resuspended in 20 μl 5% acetonitrile/0.05% formic acid prior to liquid chromatography-triple quadrupole mass spectrometry (UPLC/tandem MS) analysis (Xevo, Waters). The ¹³C₆-leucine/¹²C₆-leucine ratio in SOD1 peptides was quantified by comparing the area under the curve for the SOD1 peptides KADDLGKGGNEE (SEQ ID NO: 3), GLHGFHVHE (SEQ ID NO: 4), and SNGPVKVWGSIKGLTE (SEQ ID NO: 5) in the presence or absence of ¹³C₆-leucine.

Plasma-free ¹³C₆-leucine enrichment was quantified and used to reflect the precursor pool enrichment for SOD1 protein synthesis. Plasma proteins were precipitated with 10% trichloroacetic acid overnight at 4° C., the protein pellet was retained for quantification of bound ¹³C₆-leucine, and the supernatant was removed after centrifugation at 21,000 g for 10 minutes. The supernatant was chemically derivatized to form the N-heptafluorobutyryl n-propyl esters of plasma-free amino acids, and ¹³C₆-leucine enrichment was quantified using capillary gas chromatography-negative chemical ionization-quadrupole mass spectrometry (GC-MS; Agilent 6890N Gas Chromatograph and Agilent 5973N Mass Selective Detector) with the m/z of 355 as compared with 349 as described previously (Potter R, et al. Increased in vivo amyloid-β42 production, exchange, and loss in presenilin mutation carriers. Sci Transl Med. 2013; 5(189):189ra177; Reeds D N, Cade W T, Patterson B W, Powderly W G, Klein S, Yarasheski K E. Whole-body proteolysis rate is elevated in HIV-associated insulin resistance. Diabetes. 2006; 55(10):2849-2855). Protein-bound ¹³C₆-leucine abundance was quantified in the TCA-precipitated proteins after sonicating the pellet in a cold 10% TCA solution twice. The pellets were hydrolyzed in 6 N HCl for 24 hours at 110° C. The hydrolysates were subjected to cation-exchange chromatography (50W-X8 resin, Sigma-Aldrich) to trap the protein-bound amino acids that were eluted from the column with 6 N NH₄OH. The samples were then dried under vacuum and processed for GC-MS analysis as described previously (Parise G, Mihic S, MacLennan D, Yarasheski K E, Tarnopolsky M A. Effects of acute creatine monohydrate supplementation on leucine kinetics and mixed-muscle protein synthesis. J Appl Physiol. 2001; 91(3):1041-1047.). Labeled/unlabeled ratios from both the UPLC/tandem MS and GC-MS were obtained as tracer/tracee ratios (TTR) and converted to mole fraction label (MFL) to account for the bias that occurs at high stable tracer enrichments using the following equation: MFL=TTR/1+TTR.

¹³C₆-leucine abundance in human plasma (50 μl) and CSF (1 ml) protein precipitates (10% TCA) was also quantified. Labeled (containing ¹³C₆-leucine) and unlabeled media were prepared from RPMI-1640 medium without arginine, leucine, lysine, and phenol red (Sigma-Aldrich R-1780) supplemented with 10% dialyzed fetal bovine serum (Sigma-Aldrich F0392), 200 mg/l L-arginine, 40 mg/l lysine, and 50 mg/l of either ¹³C₆-leucine (labeled) or unlabeled leucine. HEK293T cells, which constitutively express human SOD1, were used to prepare ¹³C₆-leucine-labeled SOD1 standards. Cells were grown to near confluence in complete RPMI-1640 medium supplemented with 10% fetal bovine serum, then split into 10-cm dishes at a low concentration and allowed to settle overnight. Cells were then washed once with PBS and grown to near confluence (approximately 72 hours) in 0%, 0.08%, 0.17%, 0.34%, 0.68%, 1.25%, 2.5%, 5%, 10%, or 20% labeled/unlabeled media. Cells were then washed once with PBS and homogenized in cold lysis buffer (50 mM Tris, pH 8, 150 mM NaCl, 1% NP-40, protease inhibitors) with sonication (20% power for 20 seconds). The lysate was spun at 15,000 g for 5 minutes, the supernatant was collected, and protein was quantified using the BCA Protein Assay (Pierce). Aliquots of lysate were frozen at −80° C.

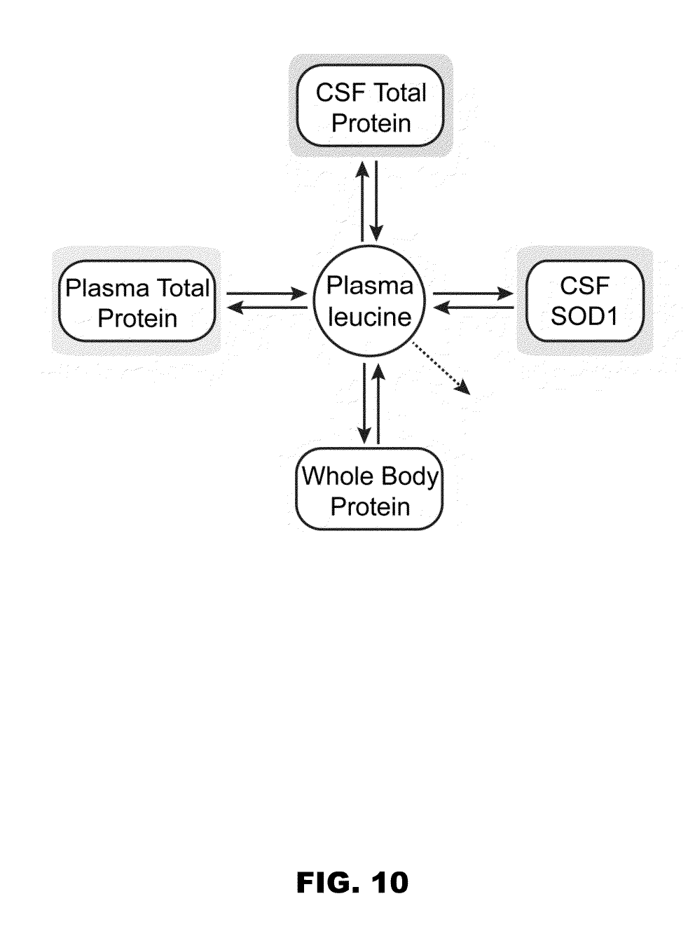

A compartmental model was developed to account for plasma-free ¹³C₆-leucine, CSF total protein, and CSF SOD1 species for each subject following 10 days of thrice-daily oral tracer administration (FIG. 10). Modeling was conducted using SAAM II (Resource for Kinetic Analysis, University of Washington, Seattle, Wash., USA). The GLHGFHVHE peptide (SEQ ID NO: 4) gave the most robust LC/tandem MS signal for the human in vivo tracer kinetics and was therefore used for modeling. The model consisted of a central plasma leucine compartment that initially received the orally administered tracer and which exchanged tracer-labeled leucine with all proteins throughout the body. Arrows connecting the compartments of the model represent first-order rate constants (units: pools per day) that describe the flux of leucine between compartments. The model describes the complete time course of tracer incorporation and clearance into each measured compartment; the FTR (pools per day) for each compartment is the rate constant for the return of leucine from each compartment back to plasma. Note that isotopic enrichment time course data were available for plasma leucine, plasma total protein, CSF total protein and CSF SOD1. A “whole-body protein” compartment was used to account for most of the shape information of plasma leucine, as label exchanged with all other unsampled proteins. The SAAM program devised first-order linear differential equations, as dictated by the structure of the model, and optimized the fit of the model-projected solution to the data for all sampled protein/tissue compartments simultaneously by adjusting the model rate constants through an iterative process. The model was set up in SAAM in a manner to simulate the appearance of label into plasma as a result of the thrice-daily tracer-dosing scheme for the 10 days of oral tracer labeling. Half-life was calculated as In 2/FTR.

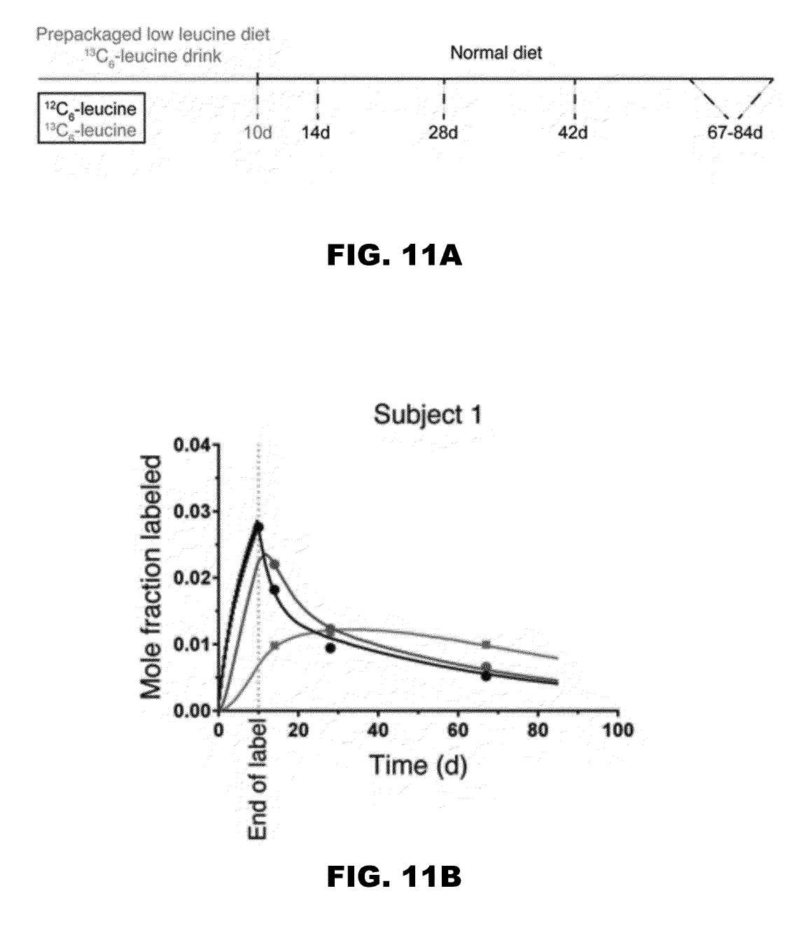

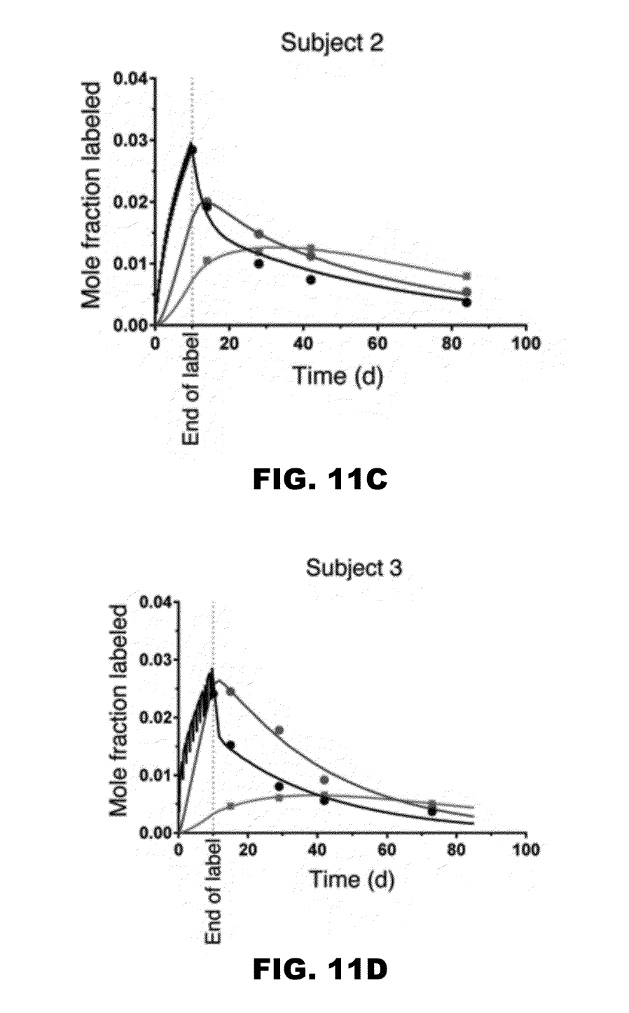

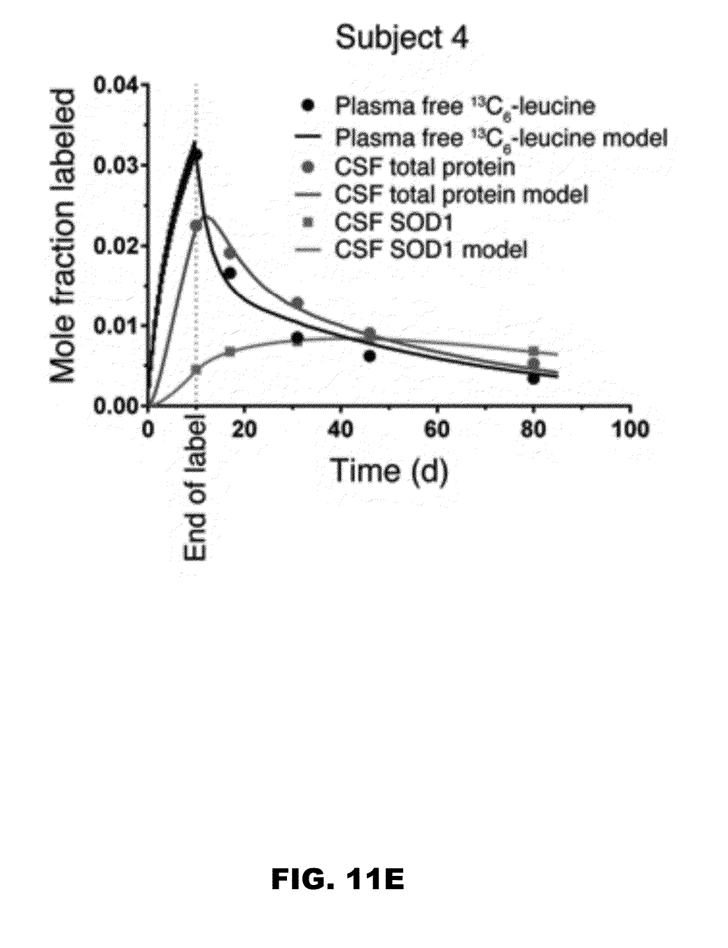

In all subjects, plasma-free ¹³C₆-leucine enrichment achieved approximately 3% at the end of label administration. This indicated that the 10-day oral labeling strategy resulted in detectable and sufficient plasma ¹³C₆-leucine enrichment for quantifying in vivo CSF SOD1 kinetics in the context of a controlled leucine diet. This was confirmed after quantifying ¹³C₆-leucine abundance in CSF total protein (Table 1). For each subject, the CSF SOD1-labeling curve displayed a slow rise and fall relative to total CSF protein labeling, indicating a much slower CSF SOD1 turnover rate of CSF SOD1 compared with that of CSF total protein. The average CSF SOD1 and CSF total protein half-life was 25.0±7.4 days and 3.6±1.0 days, respectively. Thus, the human CSF SOD1 turnover rate agreed with the slow SOD1 turnover rate observed in the rodent study. Importantly, these 4 human studies validated the ¹³C₆-leucine oral administration paradigm for quantifying long-lived protein kinetics. Data are shown in FIG. 11, and the table below.

TABLE 1
CSF SOD1 and CSF total protein half-life in healthy humans.

| | | | | | Average |
|---|---|---|---|---|---|
| | Subject 1 | Subject 2 | Subject 3 | Subject 4 | (±SD) |
| CSF total | 2.4 | 4.9 | 3.7 | 3.45 | 3.6 ± 1.0 |
| protein | | | | | |
| CSF SOD1 | 19.2 | 18.3 | 32.9 | 29.8 | 25.0 ± 7.4 |

What is claimed is:

1. A method for measuring the in vivo metabolism of SOD1 in a subject, the SOD1 being synthesized in the central nervous system, the method comprising:
(a) administering a labeled moiety to the subject, the labeled moiety being capable of crossing the blood brain barrier and incorporating into SOD1 as the SOD1 is synthesized in the central nervous system of the subject;
(b) obtaining at least one biological sample from the subject, the biological sample comprising a SOD1 fraction labeled with the moiety and a SOD1 fraction not labeled with the moiety; and
(c) detecting the amount of labeled SOD1 and the amount of unlabeled SOD1, wherein the ratio of labeled SOD1 to unlabeled SOD1 is directly proportional to the metabolism of SOD1 in the subject.

2. The method of claim 1, wherein the labeled moiety is an atom, or a molecule with a labeled atom.

3. The method of claim 2, wherein the atom is a radioactive isotope.

4. The method of claim 2, wherein the atom is a non-radioactive isotope.

5. The method of claim 4, wherein the non-radioactive isotope is selected from the group consisting of ²H, ¹³C, ¹⁵N, ¹⁷O, ¹⁸O, ³³S, ³⁴S, and ³⁶S.

6. The method of claim 1, wherein the labeled moiety is administered to the subject orally.

7. The method of claim 1, wherein the biological sample is cerebral spinal fluid.

8. The method of claim 1, further comprising separating the labeled SOD1 fraction and the unlabeled SOD1 fraction from the biological sample.

9. The method of claim 8, wherein the protein is separated by immunoprecipitation.

10. The method of claim 8, wherein the amount of labeled SOD1 and the amount of unlabeled SOD1 is detected by mass spectrometry.

11. The method of claim 1, wherein the subject is a rodent.

12. The method of claim 11, wherein the mammal is a human.

13. A kit for measuring the metabolism of SOD1 in a subject, the kit comprising:
(a) a labeled amino acid;
(b) means for administering the labeled amino acid to the subject, whereby the labeled amino acid is capable of crossing the blood brain barrier and incorporating into and labeling SOD1 as SOD1 is being synthesized in the central nervous system of the subject;
(c) means for obtaining a biological sample at regular time intervals from the subject, the biological sample comprising a labeled SOD1 fraction and an unlabeled SOD1 fraction; and
(d) instructions for detecting and determining the ratio of labeled to unlabeled SOD1 over time so that a metabolic index may be calculated, whereby the metabolic index may be compared to the metabolic index of a normal, healthy individual or compared to a metabolic index from the same subject generated at an earlier time.

14. The kit of claim 13, wherein the neurological or neurodegenerative disease is Amyotrophic Lateral Sclerosis.

15. The kit of claim 13, wherein the SOD1 synthesized is from a neuronal cell, glial cell, or other cell in the central nervous system.

16. The kit of claim 13, wherein the labeled amino acid has a radioactive or a non-radioactive atom.

17. The kit of claim 16, wherein the non-radioactive atom is selected from the group consisting of ²H, ¹³C, ¹⁵N, ¹⁷O, ¹⁸O, ³³S, ³⁴S and ³⁶S.

18. The kit of claim 17, wherein the amino acid is leucine and the non-radioactive atom is ¹³C.

19. The kit of claim 13, wherein the biological sample is cerebral spinal fluid.

20. The kit of claim 13, wherein the ratio of labeled SOD1 to unlabeled SOD1 is determined from the amounts of labeled and unlabeled SOD1 detected by mass spectrometry.


SEQUENCE LISTING (5 sequences)

SEQ ID NO: 1 - protein, 7 aa, Artificial Sequence
  BASED ON RATTUS
TLVVHEK

SEQ ID NO: 2 - protein, 12 aa, Artificial Sequence
  BASED ON HOMO SAPIENS
HVGDLGNVTADK

SEQ ID NO: 3 - protein, 12 aa, Artificial Sequence
  SYNTHESIZED
KADDLGKGGNEE

SEQ ID NO: 4 - protein, 9 aa, Artificial Sequence
  SYNTHESIZED
GLHGFHVHE

SEQ ID NO: 5 - protein, 16 aa, Artificial Sequence
  SYNTHESIZED
SNGPVKVWGSIKGLTE